(19) United States
(12) Patent Application Publication
(10) Pub. No.: US 2025/0368510 A1
(43) Pub. Date: Dec. 4, 2025

(54) Methods and Systems for Removing Contaminants from Gas

(71) Applicant: Third Pole, Inc., Waltham, MA (US)

(72) Inventors: Christopher Miles, Acton, MA (US); Frank Heirtzler, Londonderry, NH (US); Gregory W. Hall, Belmont, MA (US)

(73) Assignee: Third Pole, Inc., Waltham, MA (US)

(21) Appl. No.: 19/298,341

(22) Filed: Aug. 13, 2025

Related U.S. Application Data
(63) Continuation of application No. PCT/US2024/015866, filed on Feb. 14, 2024.
(60) Provisional application No. 63/484,783, filed on Feb. 14, 2023.

(51) Int. Cl. C01B 21/24; B01D 53/22; B01D 69/04; B01J 19/00; B01J 19/08

(52) U.S. Cl. CPC C01B 21/24; B01D 53/227; B01D 69/04; B01J 19/0006; B01J 19/088; B01D 2257/102; B01D 2257/104; B01D 2257/404; B01J 2219/00164; B01J 2219/00186; B01J 2219/0801; B01J 2219/0809; B01J 2219/0883; B01J 2219/0896

(57) ABSTRACT

Systems and methods are provided for generating a nitric oxide (NO) gas. A plasma generating device is configured to produce a plasma to ionize a flow of a reactant gas into a product gas that comprises NO, NO₂, oxygen, and nitrogen gases. A controller is configured to regulate an amount of NO in the product gas using parameters as input to the controller. A gas separation device comprising a housing including product gas inlets and sweep fluid inlets to receive a flow of the product gas and a flow of the sweep fluid such that the flows of product gas and sweep fluid are opposed flows. A membrane is positioned inside the housing and permits flow of a subset of gases of the product gas therethrough so the product gas exiting the housing includes NO.

### RELATED APPLICATIONS

This application is a continuation of PCT International Application No. PCT/US2024/015866, filed Feb. 14, 2024, which claims the benefit of and priority to U.S. Provisional Application No. 63/484,783, filed Feb. 14, 2023, each of which are incorporated herein by reference in their entirety.

### FIELD

The present disclosure relates to systems and methods for generating nitric oxide and separating gases in the nitric oxide gas flow.

### BACKGROUND

Nitric oxide gas in the presence of oxygen oxidizes to form nitrogen dioxide, a harmful chemical when inhaled. Medical devices generating and delivering nitric oxide are designed to minimize the amount of nitrogen dioxide delivered to a patient. Soda lime is a common scrubbing material utilized on the market today for the removal of NO₂ from a gas stream. Soda lime functions by providing moisture that water-soluble NO₂ enters, forming nitric acid. Once in solution, the nitric acid is neutralized by the highly alkaline constituents of the soda lime. While this is a highly effective means to remove NO₂ from a gas stream, it has finite life and requires periodic soda lime replacement to maintain efficacy as the water and hydroxide compounds are depleted.

An additional characteristic of soda lime scrubbers is that they work best when the gas path through them has high surface area and tortuosity. Thus, soda lime scrubbers can present a flow restriction to gas flow. Flow restrictions require additional energy to flow gas often resulting in larger pumps and batteries. Flow restrictions also introduce delay into the flow of product gas, slowing the step-response of a NO delivery system and potentially affecting dose accuracy at the patient. Soda lime is also a brittle material, known for releasing fine particulate that have the potential to induce patient harm when inhaled. The fine particulate can be removed by appropriate filtration approaches but not without further additions to flow restriction and system dead volume.

A NO₂ scrubber solution that significantly reduces the flow restriction over a soda lime scrubber could enable simpler NO generation architectures that would otherwise not provide accurate enough dose control.

### SUMMARY

The present disclosure is directed to systems, methods and devices for nitric oxide (NO) generation and delivery, including the use of one or more gas separators for separating components of an NO-containing gas.

A nitric oxide (NO) delivery system is provided, and in some embodiments includes a plasma generating device configured to produce a plasma to ionize a flow of a reactant gas into a product gas, the product gas including NO, NO₂, oxygen, and nitrogen gases, a controller configured to regulate an amount of NO in the product gas using one or more parameters as input to the controller, the one or more parameters including information related to at least one of the reactant gas, the product gas, a flow of gas into which the product gas is configured to be delivered, and a sweep fluid for removing scrubbed gases from the product gas; and a gas separation device including: a housing including one or more product gas inlets and one or more sweep fluid inlets, the one or more product gas inlets being configured to receive a flow of the product gas, the one or more sweep fluid inlets being configured to receive a flow of the sweep fluid such that the flow of product gas and the flow of the sweep fluid are opposed flows, and at least one membrane positioned inside the housing. The at least one membrane is configured to permit flow of a subset of gases of the product gas therethrough such that the product gas exiting the housing includes NO. The flow of the sweep fluid through the housing is configured to move a subset of gases separated from the product gas away from the at least one membrane.

In some embodiments, the sweep fluid is configured to pass through a filter before entering at least one of the one or more sweep fluid inlets of the housing of the gas separation device. In some embodiments, the sweep fluid is configured to exit the housing through an outlet, the sweep fluid configured to pass through a filter after exiting the housing of the gas separation device.

In some embodiments, the system further includes one or more pumps upstream of the housing configured to apply a pressure gradient across the at least one membrane to promote gas transport across the at least one membrane.

In some embodiments, the at least one membrane includes a plurality of tubular membranes to provide a plurality of product gas pathways through the gas separation device. In some embodiments, the plurality of tubular membranes are positioned in parallel to provide even flow restriction therethrough.

In some embodiments, a flow of product gas exiting the housing is configured to flow to one of the one or more product gas inlets to provide a recirculation flow of product gas through the gas separation device. In some embodiments, a flow of the sweep fluid exiting the housing is configured to flow to one of the one or more sweep fluid inlets to provide a recirculation flow of the sweep fluid through the gas separation device. In some embodiments, the sweep fluid is reactant gas.

In some embodiments, the plasma generating device includes at least one pair of electrodes. In some embodiments, the controller is configured to regulate the amount of NO in the product gas by using the one or more parameters to control sparking of the at least one pairs of electrodes. In some embodiments, the controller is configured to regulate the amount of NO in the product gas by using the one or more parameters to control energy transferred to the plasma.

In some embodiments, the system further includes a sensor configured to measure a flow of the sweep fluid, the sensor configured to communicate the measured flow of the sweep fluid to the controller such that the controller suspends plasma production when the measured flow of the sweep fluid is below a threshold.

In some embodiments, the gas separation device is in the form of a replaceable cartridge.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 depicts the effect of temperature on solubility of various gases in water

FIG. 2 is an exemplary embodiment of a membrane scrubber;

FIG. 3 is an exemplary embodiment of a membrane scrubber with sweep flow;

FIG. 4 is an exemplary embodiment of a membrane scrubber with turbulence elements;

FIG. 5 is an exemplary embodiment of a membrane scrubber with a membrane with a tubular shape;

FIG. 6 is an exemplary embodiment of a membrane scrubber with a partition and multiple sweep gas ports;

FIG. 7 is an exemplary embodiment of a membrane scrubber with partition and common sweep gas outlet;

FIG. 8 is an exemplary embodiment of an unconstrained convective sweep flow acting in membrane scrubber tubes;

FIG. 9 is an exemplary embodiment of a membrane scrubber with uniform flow paths for product gas and sweep gas;

FIG. 10 is an exemplary embodiment of a membrane scrubber with helical sweep gas flow path;

FIG. 11 is an exemplary embodiment of a membrane scrubber with static fluid;

FIG. 12 is an exemplary embodiment of a membrane scrubber system with sweep fluid reservoir and waste reservoir;

FIG. 13 is an exemplary embodiment of a NO generation system with recirculation architecture and a membrane scrubber;

FIG. 14 is an exemplary embodiment of a NO generation system with filtration and scrubbing of sweep fluid;

FIG. 15 is an exemplary embodiment of a pulsed NO generation system with a membrane scrubber;

FIG. 16 is an exemplary embodiment of a pulsed NO generation system that utilizes reactant gas flow to scrub product gas;

FIG. 17 is an exemplary embodiment of a linear architecture NO system with flow-restricted scrubber sweep flow;

FIG. 18 is an exemplary embodiment of a membrane scrubber that collects NO in the sweep gas;

FIG. 19 is an exemplary embodiment of a magnetic gas separator with opposed magnets;

FIG. 20 is a cross-sectional view of an exemplary embodiment of a magnetic gas separator with spiral gas flow path;

FIG. 21 is a top view of an exemplary magnetic gas separator with spiral gas flow path;

FIG. 22 is a cross-sectional view of an exemplary embodiment of a magnetic gas separator with spiral gas flow path and stacked magnets;

FIG. 23 is an exploded view of a magnetic gas separation device;

FIG. 24 is an embodiment of a NO generation system that utilizes a magnetic gas separation device;

FIG. 25 is an exemplary embodiment of a system for separating gas constituents using centripetal acceleration;

FIG. 26 is an exemplary embodiment of a system that purifies a NO product gas stream using the Hampson-Linde process; and

FIG. 27 is an exemplary embodiment of a system that variably purifies a NO product gas stream using vacuum pressure;

FIG. 28 is an exemplary embodiment of a system for generating a NO-enriched product gas;

FIG. 29 is an exemplary embodiment of a system for generating a NO-enriched product gas; and

FIG. 30 is an exemplary embodiment of a system for generating a NO-enriched product gas that utilizes a recirculation architecture.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments.

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail in order to avoid obscuring the embodiments. Figures depicting architectures forgo the details of also depicting cabling and control elements to provide focus on the innovation.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The subject disclosure applies to the field of nitric oxide (NO) generation which commonly creates nitrogen dioxide as a byproduct. This disclosure utilizes one or more membranes to draw a first type of gas away from a second type of gas without consumption of the membrane material itself. For example, nitrogen dioxide (NO₂) can be drawn away from a NO product gas. In some embodiments, this can be achieved by allowing the NO₂ to pass through the scrubber membrane. Typical sweep fluids (i.e., the material that carries the NO₂ away from the product gas) are typically common gases and liquids that are easily sourced and disposed of. Various designs and techniques are presented to affect the scrubber performance and longevity.

A gas separator can be provided to separator different components of gases, or a scrubber can be provided as a means to scrub one or more gases from a NO gas stream, to prolong the longevity of the gas (i.e., remove oxygen) and/or make the gas safer to inhale (i.e., remove NO₂). In some embodiments, this separation and/or scrubbing is accomplished with a material that is not exhausted in the scrubbing process thereby enabling, in some embodiments, a permanent gas scrubber. In some embodiments, where the scrubber has a finite life, for example when the membrane material decreases its efficiency over time, there can still be decreased flow restriction and particulate generation. A gas exchange membrane can be utilized that permits the transfer of one gas (for example, NO₂) more than the other gas (for example, NO). It will be understood that the membrane can be configured to allow for any gas to transfer more than another gas. For example, the membrane can permit the transfer of NO and/or other gases more than NO₂. In some embodiments, a sweep fluid (e.g., gas or liquid) carries scrubbed NO₂ away from the membrane. In some embodiments, a static fluid on one side of the membrane absorbs NO₂ and is replaced periodically. The scrubbing effect can be enhanced with a pressure gradient across the membrane, NO₂-containing gas flow rate, and sweep fluid flow rate. This approach to gas separation can provide benefits in flow restriction, simplicity, and operating cost over the life of a NO generation/delivery device when compared to soda lime scrubbers.

The present disclosure relates to systems and methods of nitric oxide (NO) delivery for use in various applications, for example, inside a hospital room, in an emergency room, in a doctor's office, in a clinic, and outside a hospital setting as a portable or ambulatory device or gas source during patient transport. An NO generation and/or delivery system can take many forms, including but not limited to a device configured to work with an existing medical device that utilizes a product gas, a stand-alone (ambulatory) device, a module that can be integrated with an existing medical device, one or more types of cartridges that can perform various functions of the NO system, a compact NO inhaler, and an electronic NO tank. In some embodiments, the NO generation system uses a reactant gas containing a mixture of at least oxygen and nitrogen, including but not limited to ambient air, and an electrical discharge (plasma) to produce a product gas that is enriched with NO. In some embodiments, the NO generation system generates NO from a source material (e.g., N₂O₄) or releases NO from a donor material.

An NO generation device can be used with any device that can utilize NO, including but not limited to a ventilator, an anesthesia device, a defibrillator, a ventricular assist device (VAD), a Continuous Positive Airway Pressure (CPAP) machine, a Bilevel Positive Airway Pressure (BiPAP) machine, a non-invasive positive pressure ventilator (NIPPV), a nasal cannula application, a nebulizer, an extracorporeal membrane oxygenation (ECMO), a bypass system, an automated CPR system, an oxygen delivery system, an oxygen concentrator, an oxygen generation system, and an automated external defibrillator AED, MRI, and a patient monitor. In addition, the destination for nitric oxide produced can be any type of delivery device associated with any medical device, including but not limited to a nasal cannula, a manual ventilation device, a face mask, inhaler, or any other delivery circuit. The NO generation capabilities can be integrated into any of these devices, or the devices can be used with a NO generation device as described herein.

The present disclosure includes ideas in the areas of NO generation and NO delivery. It should be noted that NO delivery concepts can be applicable to NO delivered from a multitude of sources, including NO tanks, electrically generated NO and chemically derived NO.

In some embodiments, a membrane scrubber consists of a gas flow path bounded by one or more walls of gas-permeable membrane material. The permeability of the membrane material permits some gases through it more than others. In some embodiments, a silicone membrane material is utilized with more than 12× more permeability to NO₂ than to both NO and oxygen.

Some level of NO loss through the membrane is acceptable since there is typically much more NO than NO₂ to begin with. For example, typical NO to NO₂ ratios for electrical generation of NO are 10:1. Transport of the NO₂ through the membrane can be enhanced by increasing the pressure gradient across the membrane, increasing membrane surface area, and increased flow rate of sweep gas external to the membrane to increase the concentration gradient.

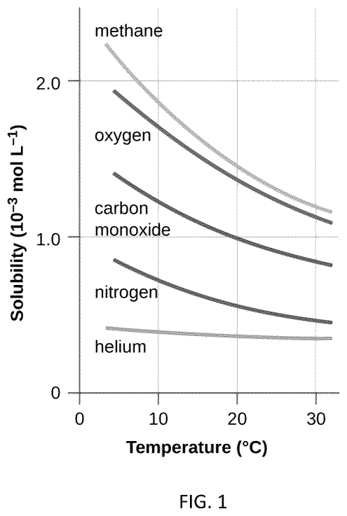

Sweep flow can be gaseous or liquid. In some embodiments, water is utilized within the scrubber as the sweep material. Water can be used as the sink material because NO₂ is soluble in water while NO is not. FIG. 1 depicts an exemplary graph showing the solubility of various gases in water as a function of temperature. In some embodiments, a sweep liquid is cooled to enhance nitrogen dioxide solubility. In some embodiments, cooling is active (e.g., thermoelectric device, compression/expansion process) while other embodiments utilize passive cooling of the fluid (e.g., cooling fins, ice cubes, etc.).

In some embodiments, still water is utilized to capture NO₂. As the system operates, NO₂ entering the water forms nitric acid and the pH of the solution will decrease. In some embodiments, an NO generation system measures pH of the sink solution and alerts a user to replace the sink solution at a particular threshold. In some embodiments, the sink solution includes a buffer that enables the system to maintain a more neutral pH for a period of time. In some embodiments, the sink solution is alkaline (pH >7) to neutralize nitric acid as it forms in solution. In some embodiments, the system replaces the sink solution automatically by opening one or more valves to control sink solution flow.

The subject discovery leverages the capabilities of various gas separation methodologies to purify a NO-containing gas stream for inhalation. In some embodiments, gas separation membranes are utilized to separate specific constituents of a gas stream. In some embodiments, the membrane consists of a monolithic material with particular permeability properties (e.g., certain varieties of silicone). In some embodiments, the membrane is a mechanical sieve with pore sizes that permit small molecules (e.g., NO) and block larger molecules (e.g., NO₂). Mechanical sieve membranes are constructed from various materials including metals (e.g., stainless steel), ceramics (e.g., zeolite through which NO and NO₂ can pass), and polymers (e.g., PTFE). In some embodiments, the membrane is a composite material that leverages the material properties of more than one material. In some embodiments, the membrane consists of a perforated substrate (e.g., PTFE) coated with a selectively gas-permeable material (e.g., silicone). The substrate provides structural integrity while the gas-permeable material provides selective gas transport properties. In other embodiments, the substrate consists of a woven or non-woven textile that is coated with a selective gas-transport material. In some embodiments, the membrane is wetted to absorb NO₂ and not NO. In some embodiments, the membrane consists of multiple layers of material. In some embodiments, various layers are selected for specific separation properties. For example, a first layer blocks all gases from passing through except for NO and a second gas and a second layer to the membrane permits NO to pass and blocks the second gas. By stacking layers of membranes, the membrane can have a variety of structural and gas-selective properties, as required.

In some embodiments, a NO-selective membrane can be constructed with protein channels (e.g., connexin) that transport NO. Protein channels are utilized by living cells to transport NO across a cell membrane. NO permeates readily through connexin channels 43, 40, and 37, and possibly others. In some embodiments, protein channels are fixated into an artificial amphiphilic block copolymer to provide preferential/selective NO diffusion.

Sweep gas can be used to collect NO from the membrane, and the sweep gas can be used for various purposes and be composed of a variety of different gases/molecules. In some embodiments, the NO-loaded sweep gas is flowed directly to a patient for inhalation. In other words, the sweep gas flow can be used as the inspiratory flow to a patient. In this case, the sweep gas can be air or another oxygen-containing gas. In some embodiments, the sweep gas has low oxygen levels or is devoid of oxygen (e.g., N₂) which enables the NO-containing sweep gas to be stored for a period of time before delivery to a patient. In some embodiments, the NO-containing sweep gas is diluted prior to delivery to a patient to achieve a target oxygen concentration and/or achieve a target NO concentration. In some embodiments, the sweep gas consists of an inert gas (nitrogen, helium, argon, etc.). This can be used to dilute the NO product gas that passed through a membrane so that the decreased concentration reduces NO oxidation.

In some embodiments, the membrane material is composed of polydimethylsiloxane (PDMS). PDMS material is not compatible with nitric acid, however by utilizing either a buffered solution or an alkaline solution, any nitric acid formed by NO₂ in water is quickly neutralized, thereby prolonging the service life of the membrane.

In some embodiments, the membrane is composed of one or more of polymethylpentene, polypropylene, and polyester. In some embodiments, the membrane is comprised of a metal organic framework material (e.g., UiO-66, UiO-66, MFM-520), which functions as a trap for molecules rather than as a molecular sieve.

In some embodiments, a scrubber is comprised of a substrate gas-permeable material coated with TEMPO or a TEMPO variant. This type of scrubber can be constructed by dissolving the TEMPO material in a mixture of ethanol and water, then dipping the substrate material (e.g., fiber, woven or non-woven fabric, open-cell, foam, etc.) into the dissolved TEMPO. After dipping, solvent is driven off (e.g., with heat and or convection), leaving a TEMPO coating on the substrate. In some embodiments, the scaffold is electrically conductive, providing the ability to apply a voltage to the TEMPO material in order to release captured NO₂ and reset the membrane.

A membrane can be configured to selectively allow certain molecules to pass through while leaving other molecules in the scrubber. In some embodiments (not shown), NO and optionally nitrogen and/or oxygen pass through the membrane, leaving the larger NO₂ molecules in the primary flow. In some embodiments, only NO and nitrogen pass through the membrane. This eliminates the potential for NO to oxidize. In some embodiments, only O₂ passes through the membrane, thereby leaving NO in a nitrogen-rich gas to prevent further oxidation.

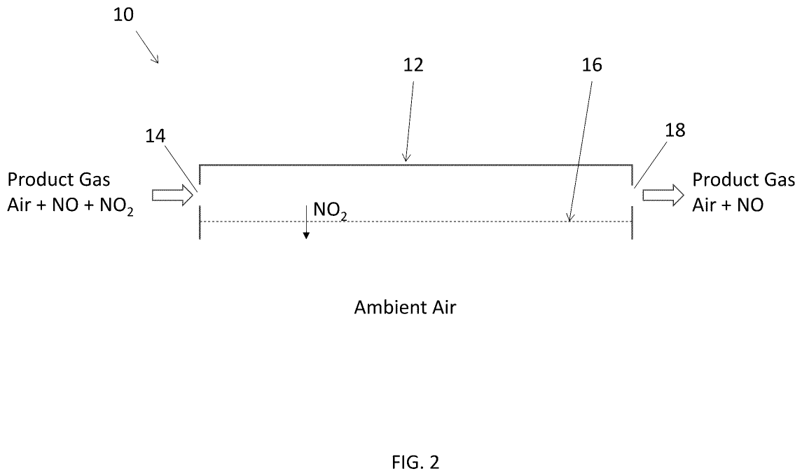

FIG. 2 depicts an exemplary embodiment of a membrane gas scrubber 10. Product gas containing a contaminant (e.g., NO₂) passes into the enclosure or housing 12 of the scrubber 10, for example, through an inlet 14. The product gas flow path passes over a membrane 16 within the scrubber enclosure or housing. On the other side of the membrane is ambient air. In some embodiments, a contaminant within the product gas (e.g., NO₂) permeates the membrane 16 more than another desirable gas material (e.g., NO). The air has a lower if not zero concentration of the contaminant, creating a concentration gradient across the membrane. The contaminant passes through the membrane into ambient air, where it is diluted. The gas exiting the housing 12, for example, through an outlet 18, can be in the form of the product gas that has been scrubbed of the contaminant gas.

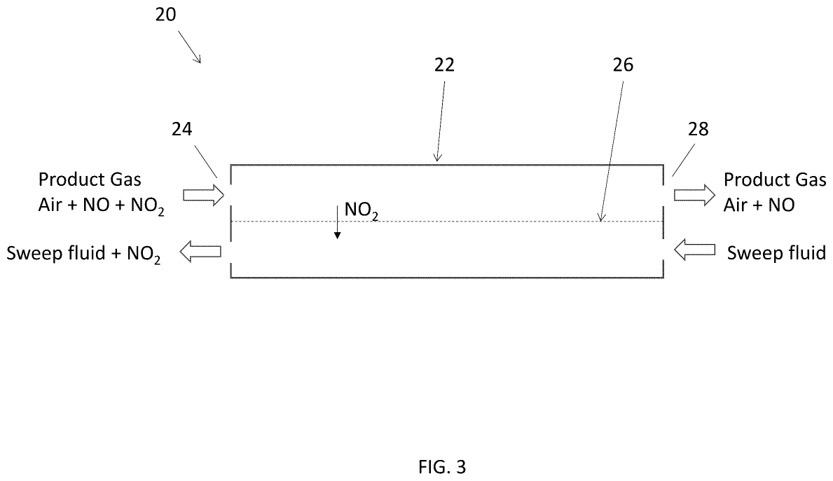

FIG. 3 depicts an exemplary embodiment of a membrane gas scrubber 20 that is similar to the embodiment of FIG. 2, with the addition of a convective flow of sweep fluid across the membrane 26 disposed in the housing 22. Product gas enters the housing 22 through a first sidewall 24 and sweep fluid enters the housing 22 through a second opposed sidewall 28 of the housing and flows counter to the product gas along the lower surface of the membrane 26 to maximize the concentration gradient across the membrane for the length of the membrane. In some embodiments, the pressure of the contaminated gas is higher than ambient pressure to create a pressure gradient across the membrane to assist in driving the contaminant material across the membrane.

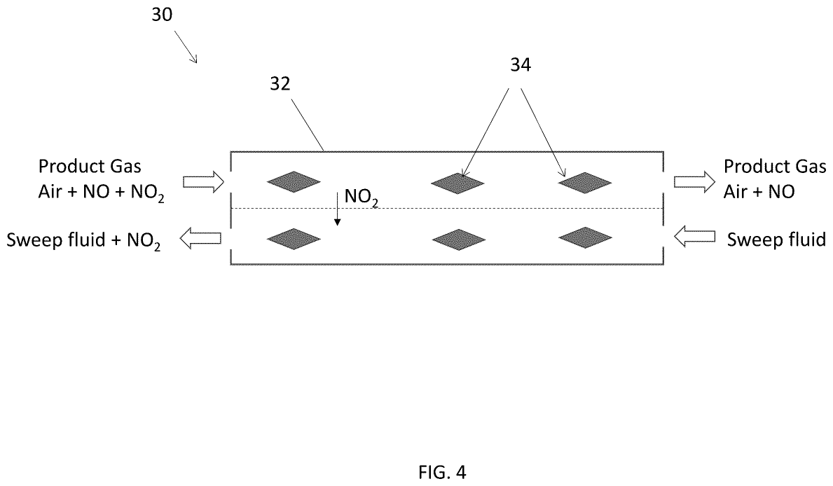

In some embodiments, a membrane gas scrubber can include one or more mixing elements in the gas flow paths. FIG. 4 depicts an embodiment of a membrane gas scrubber 30 with one or more mixing elements 34 in the flow paths through the housing 32 of the scrubber 30. The one or more mixing elements 34 induce turbulence in the product gas and sweep fluid to move NO₂ in the product gas towards the membrane and NO₂-loaded sweep fluid away from the membrane, thereby improving NO₂ transfer. It will be understood that one or more mixing elements can be used with any of the embodiments described herein.

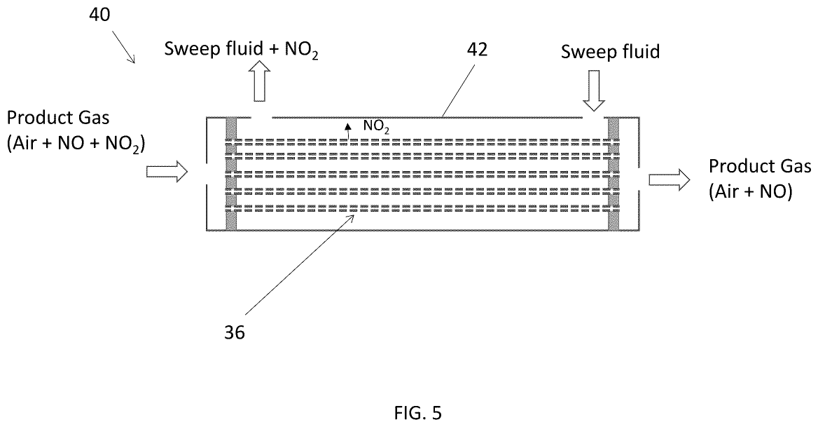

In some embodiments, a membrane gas scrubber can include a plurality of tubes composed of a membrane material through which the product gas can flow. FIG. 5 depicts an embodiment of a membrane gas scrubber 40 that utilizes one or more membrane tubes 36 constructed of membrane material that run along the length of a housing 42. Sweep fluid passes over the membrane material tubes to draw NO₂ through the walls of the tubes as product gas passes through the center of the tubes. A tubular design allows for improved surface area to product gas volume ratio. Volume of product gas within the scrubber can be a concern because it affects transit time through the scrubber and increased transit time equates to an increased NO oxidation time (i.e., increased NO₂ formation within the product gas).

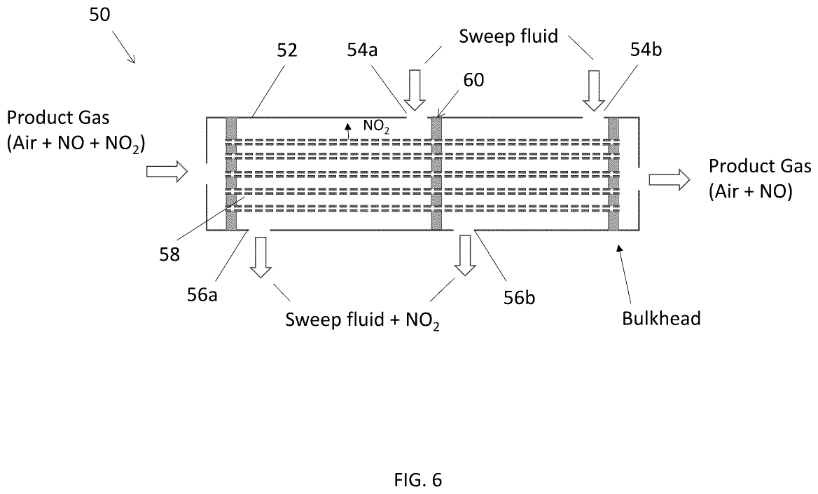

In some embodiments, a membrane gas scrubber can include a plurality of sweep gas inlets and a plurality of sweep gas outlets. FIG. 6 depicts an embodiment of a membrane scrubber 50 having a housing 52 with multiple sweep inlets 54a, 54b and multiple sweep outlets 56a, 56b. It will be understood that, while FIG. 6 illustrates two inlets and two outlets, any number of inlets and outlets can be used. This approach can provide additional scrubbing by providing fresh sweep gas at more than one location, thereby increasing the overall diffusion gradient across the one or more membranes 58. The embodiment shown in FIG. 6 includes an optional bulkhead or partition 60 at the middle of the chamber to prevent interaction between the two sweep gas flows. This solution is functionally similar to having two or more independent membrane scrubbers in series. It will be understood that the housing can include multiple bulkheads or partitions if required based on the number of inlets and outlets and the thus the number of sweep flows moving through the housing.

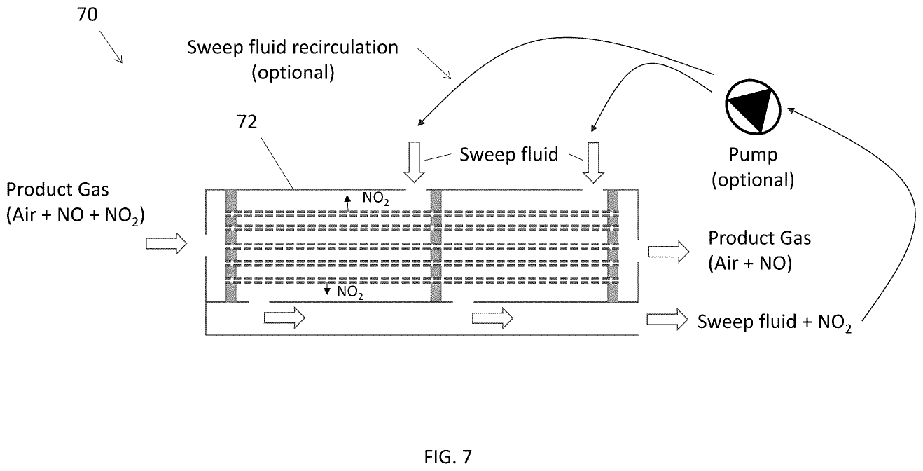

FIG. 7 depicts an embodiment of a membrane gas scrubber 70 that is similar to the embodiment shown in FIG. 6 with a difference in how used sweep fluid is collected. In some embodiments, the sweep fluid can be collected (e.g., in a container), recirculated, repurposed and/or processed (e.g., scrubbed) after flowing through the housing 72 of the membrane scrubber 70.

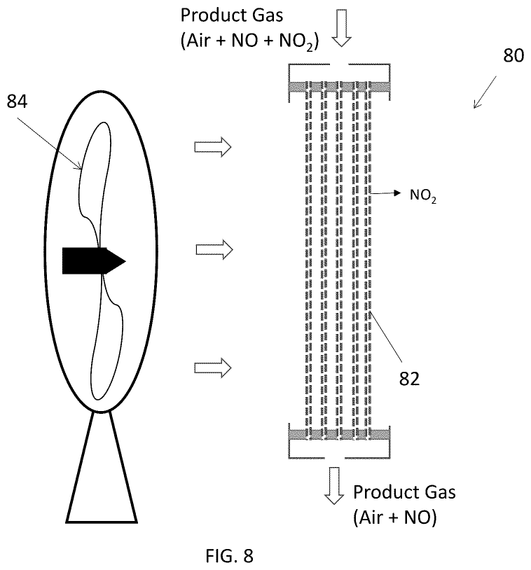

FIG. 8 depicts an embodiment of a membrane gas scrubber 80 that includes one or more membrane scrubber tubes 82 that are exposed to air. A fan 84 (or equivalent device) blows sweep gas (e.g., air) across the tubes to maintain a high concentration gradient between the contaminated gas within the tubes and the air outside the tubes.

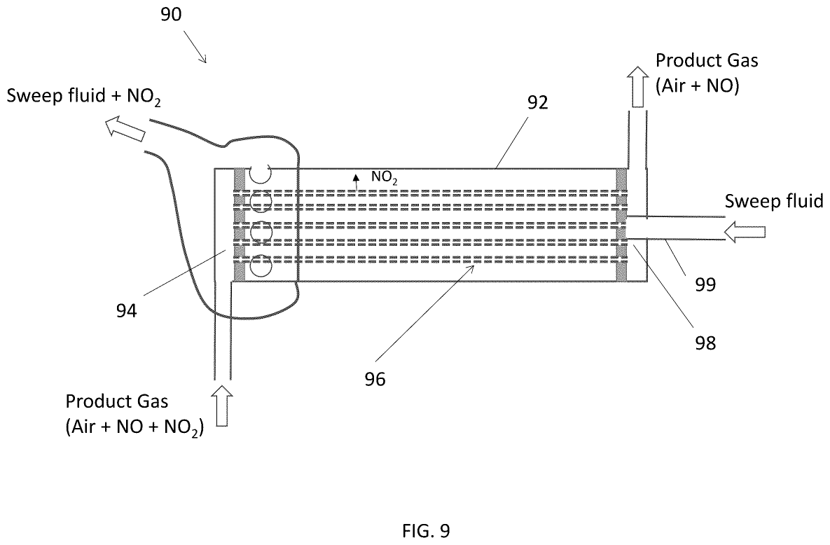

FIG. 9 depicts an embodiment of a membrane gas scrubber 90 that provides an even flow through the scrubber channels and sweep path. Product gas enters the housing 92 of the scrubber 90 into a first manifold 94 of one or more membrane tubes 96. The product gas passes through the membrane tubes and enters a second manifold 98 and exits out the device. Regardless of which membrane tube the product gas flows through, the path length is an equal total amount of travel through the scrubber. This ensures equivalent flow restriction through each membrane tube path which, in-turn, ensures equal flow through each membrane tube path. This approach prevents there being one or more tubes that are favored, resulting in faster flow through this subset of tubes, shorter transit time, and lower net scrubbing.

Sweep gas flow within FIG. 9 is also designed to be even throughout the design. Fresh sweep gas enters the scrubber housing through a sweep gas inlet 99 at the center of the membrane tube array. As sweep gas travels counter to the direction of product gas flow and the sweep gas also travels radially outward. Sweep gas is collected through an array of holes around the periphery of the sweep gas housing to provide even sweep gas flow around the perimeter of the housing. This approach allows for consistent sweep gas interaction with the membrane tubes, further improving overall scrubbing efficacy.

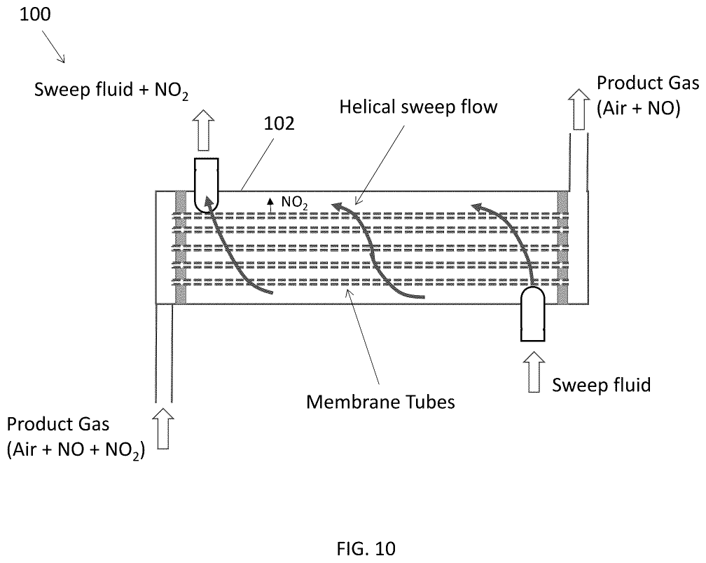

FIG. 10 depicts an embodiment of a membrane gas scrubber 100 where the sweep fluid is introduced tangent to the circumference of a cylindrical scrubber housing 102. The sweep fluid flows in a helical pattern from one end of the scrubber housing to the other end, where it exits the housing. This helical flow pattern mitigates against dead zones within the scrubber housing, where NO₂ could accumulate, and improves the consistency of scrubbing throughout the chamber.

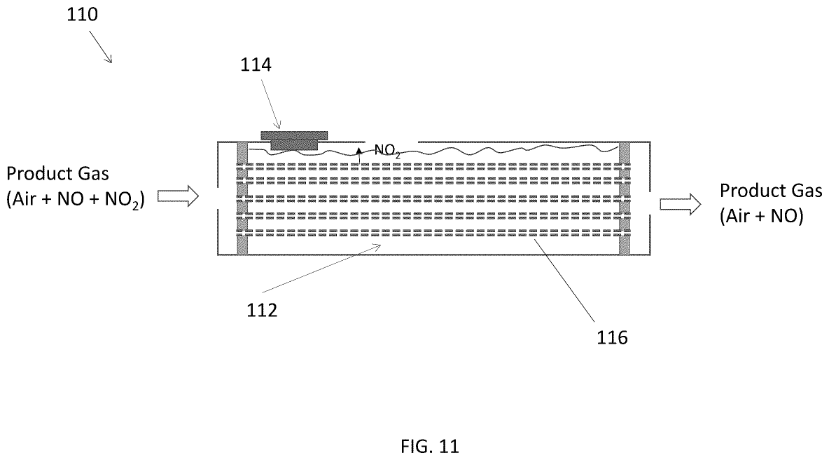

In some embodiments, the membrane gas scrubber can be positioned inside fluid filled housing. This allows for the absence of sweep flow as the static liquid surrounding the product gas flow paths acts as a sink for NO₂. FIG. 11 depicts an embodiment of a membrane gas exchange scrubber 110 with one or more tubular membranes 116 within a sealed fluid-filled enclosure or housing 112. The fluid is static within the chamber. A cap 114 can be removed to pour out used fluid and introduce new fluid. In some embodiments, the fluid is water. In some embodiments, the fluid is alkaline (e.g., metal hydroxide solution, sodium hydroxide, lye). In some embodiments, the fluid includes buffering compounds (e.g. phosphate, Tris(Hydroxymethyl)aminomethane, sodium bicarbonate, etc.) to neutralize the nitic acid that forms from NO₂ in water.

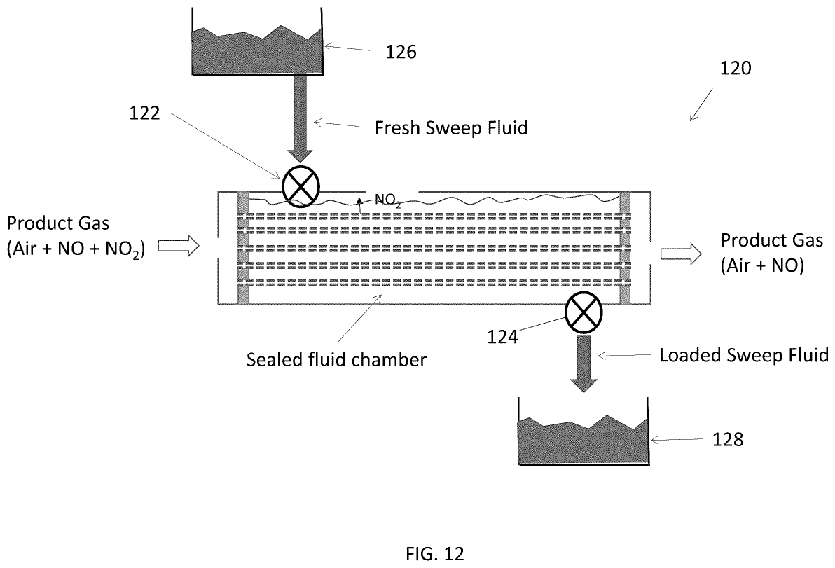

Any of the scrubber embodiments disclosed herein can optionally include features to allow for flow control of the product gas and/or sweep gas relative to the scrubber housing. FIG. 12 depicts an embodiment of a membrane gas exchange scrubber 120 with one or more valves before and after the exchange scrubber to control the flow of sweep fluid. As shown, the scrubber 120 includes an inlet valve 122 and an outlet valve 124 associated with sweep fluid. Sweep fluid is sourced from a sweep fluid source 126, such as a reservoir, and NO₂-loaded sweep fluid within the membrane scrubber is drained into a waste reservoir/fluid collector 128. The valves and corresponding flow of sweep fluid are controlled by the overall system controller in some embodiments. In some embodiments, the sweep fluid source 126, membrane scrubber 120, and waste reservoir 128 are combined into a single assembly. In some embodiments, the assembly is removable and/or disposable. In some embodiments, the valves consist of pinch valves acting on tubing that is part of the assembly. In some embodiments, the sweep fluid reservoir is a permanent component of a system and is filled by a user. In some embodiments, the waste reservoir is a permanent component of a system that is drained by a user. In some embodiments, the flow of sweep fluid is gravity-fed (as shown). In some embodiments, the flow of sweep fluid is driven by a pump.

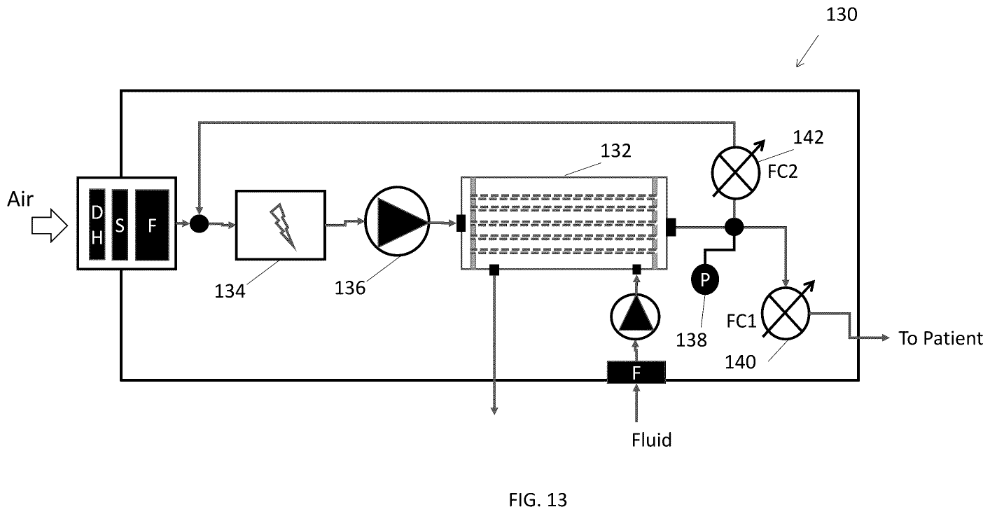

FIG. 13 depicts an embodiment of an NO generation system 130 utilizing a tubular membrane scrubber 132. The tubular membrane scrubber used with the NO generation system can be any of the embodiments described herein. The NO generation system operates by pulling in ambient air through a dehumidifier (DH), scrubber(S), and particle filter (F). The scrubber includes chemistry for removing one or more of volatile organic compounds (VOCs) and non- organic compounds (e.g., ammonia) from the incoming gas. This dried, scrubbed and filtered air, referred to as “reactant gas,” passes through a plasma chamber 134 where a plasma ionizes the nitrogen and oxygen in the reactant gas, with a fraction of the disassociated atoms forming NO and a smaller fraction forming NO₂. This reactant gas with NO and NO₂ is referred to as “product gas.” Product gas is pulled through a pump 136 and pushed through the scrubber 132. At the exit of the scrubber, the pressure of the gas is measured with a pressure sensor 138 (P). Gas flows to either the patient or to a return path, governed by flow controllers. A flow controller labeled 140 controls the flow of NO to the patient. A return flow path flow controller 142 controls the pressure at the pressure sensor to maintain a constant pressure. In some embodiments, the flow rate through the plasma chamber and pump is constant. In some embodiments, the concentration of product gas downstream of the plasma chamber is managed to be constant by varying plasma activity (frequency, duration, energy). The sweep fluid enters the system, passes through the membrane gas exchanger and exits the system, propelled by a pump. In some embodiments, as shown, the incoming fluid is filtered prior to passing through the remainder of the system. In some embodiments, when the sweep fluid is a gas, loaded sweep fluid is released back into the atmosphere. In some embodiments, the sweep fluid is directed towards a vacuum source at the facility. In some embodiments, the NO generation system includes a connector for connecting to a vacuum source.

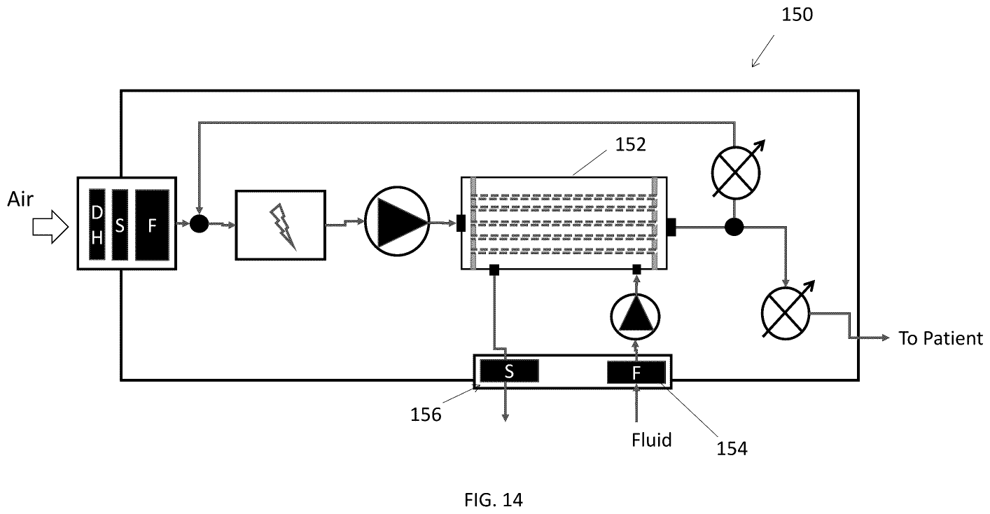

FIG. 14 depicts another embodiment of a NO generation system 150 utilizing a membrane scrubber 152. This NO generation system 150 is similar to the one shown in FIG. 13, but in this embodiment, the incoming sweep fluid (gas or liquid) is filtered using a filter 154, and the outgoing sweep fluid is scrubbed using an exit scrubber 156. Scrubbing the sweep gas enables the system to utilize the low product gas transit time achievable with membrane scrubbing. Scrubbing of the sweep gas does not have the same constraints as scrubbing of NO product gas. Thus, a sweep gas scrubber can have high dead volume, and slow flow rates for long residence times that would result in excessive NO loss (NO₂ formation) when applied to the product gas. In the depicted embodiment, the incoming filter and outgoing scrubber are packaged in a single assembly for ease of replacement.

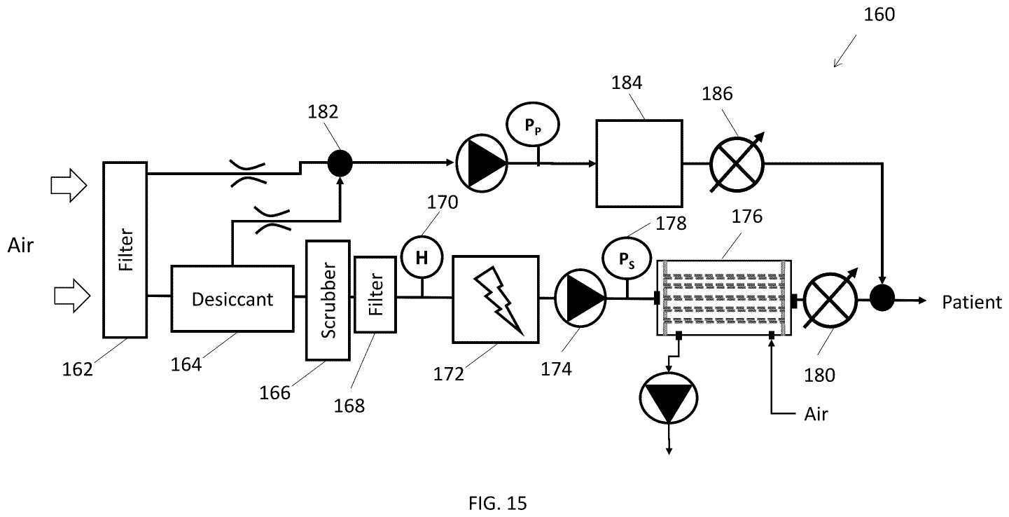

FIG. 15 depicts an embodiment of a pulsed NO generation and delivery system 160. Air enters the system and flows through a particle filter 162. The lower flow path removes water from the air with desiccant 164. A portion of the desiccated flow is mixed with the upper pathway to prevent water condensation at elevated pressure locations within the system. The ratio of desiccated air to non-treated air is fixed in this embodiment by fixed diameter orifices, as shown. In some embodiments, the mixture of dry and not dry gas is actively controlled with one or more flow controllers. The remainder of desiccated gas flows through a scrubber 166 to remove VOCs, ammonia and/or other potentially harmful materials and a particle filter 168. A humidity sensor 170 (labeled with “H”) measures water content in the air to detect exhaustion and/or failure of the desiccation feature. The reactant gas continues to flow along the lower path into a plasma chamber 172 where nitrogen and oxygen are ionized to form NO. After flowing through a product gas pump 174, the NO-containing gas flows into a membrane scrubber 176 with pressure monitored by a pressure sensor 178. The product gas flows through membrane tubes of the membrane scrubber while a sweep fluid (e.g., air) is moved by pump around the outside of the membrane tubes. A flow controller 180 at the exit of the membrane scrubber 176 controls the flow of product gas to the patient. The upper flow path travels through a pump 182 that pressurizes a reservoir 184 of partially desiccated air. The flow out of the reservoir is controlled by a flow controller 186 at the exit of the reservoir. In some embodiments, the system delivers a pulse of NO from the membrane scrubber, followed by a purge gas pulse to clear the delivery device of NO and NO₂ between breaths. Pressurizing the product gas within the membrane scrubber enables faster transport of NO₂ through the membrane into the sweep flow. Placing the sweep flow pump after the membrane scrubber can create a lower pressure in the sweep gas flow within the scrubber housing, increasing the pressure gradient across the membrane further.

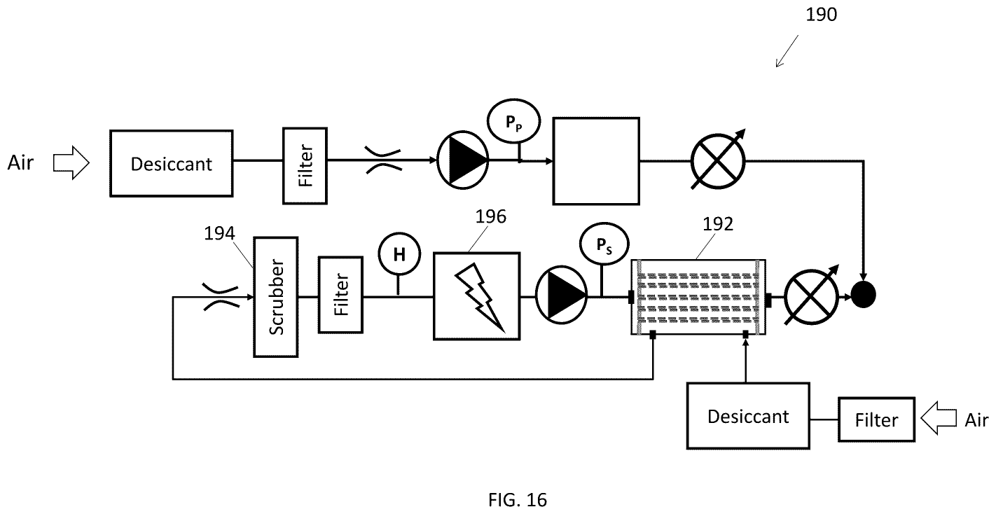

FIG. 16 depicts another embodiment of a pulsed NO generation and delivery system 190. In this embodiment, the incoming air for NO generation is utilized as the sweep gas for the membrane scrubber 192. The incoming air picks up NO₂ from the product gas and then a scrubber 194 downstream removes the NO₂ prior to entry into a plasma chamber 196. This design decreases the number of pumps required, as compared to the design presented in FIG. 15. This approach can reduce the total number of disposable scrubbers by utilizing the reactant gas scrubber to capture NO₂ from the product gas in addition to environmental contaminants. This approach can also allow for more options for reactant gas scrubber material because it is acceptable for the scrubber to scrub NO in addition to NO₂. Thus, materials like activated carbon, metal organic framework (MOF), TEMPO, and potassium permanganate can be used, which do not have the same humidity requirements that soda lime has.

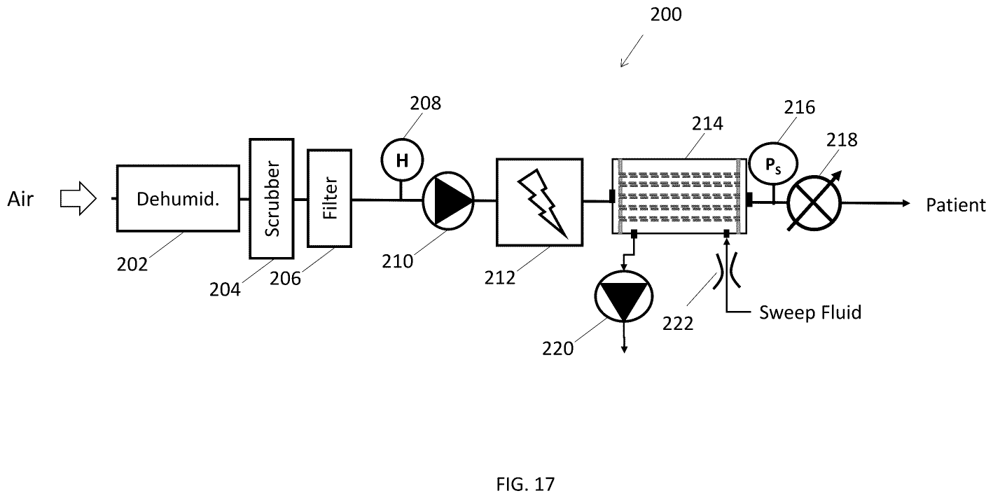

FIG. 17 depicts an embodiment of a NO generation device 200 with a linear architecture. Reactant gas containing oxygen and nitrogen enters the system and passes through a dehumidification stage 202, a chemical scrubber 204 (for VOCs, ammonia, etc.), and a particle filter 206. A humidity sensor 208 measures the incoming humidity to detect when the dehumidification stage is exhausted or non-functional. Various methods for dehumidification can be utilized alone or in combination, including by not limited to Nafion tubing, desiccant, heating, compression, cooling to induce water condensation, and other approaches. Some methods of desiccation (e.g., molecular sieve) and chemical scrubbing (e.g., activated carbon) introduce particulates into the reactant gas, thereby requiring a particle filter.

The prepared reactant gas passes through a pump 210 and into a plasma chamber 212 where NO is formed. The NO-containing product gas passes through a membrane scrubber 214. A pressure sensor 216 in fluid communication with the product gas flow path is used to detect an obstruction in the system. The pressure is also utilized as an input to a flow controller 218 to ensure that an accurate number of moles of NO are delivered to the patient out the right end of the figure. A separate pump 220 is utilized to draw sweep fluid through the membrane scrubber 214. An orifice 222 on the sweep fluid inlet restricts the sweep fluid flow, lowering the pressure within the sweep space within the membrane scrubber. This increases the pressure gradient across the membrane, enhancing transport of NO₂ into the sweep fluid.

### Magnetic Features

NO is paramagnetic, meaning that it will align with and be pulled in the direction of the magnetic field lines. NO₂ is not paramagnetic, hence its motion is not influenced by a magnetic field. This difference in magnetic properties between NO and NO₂ can be leveraged to separate NO from NO₂ within a gas stream. Magnetic fields are dictated by the shape, strength, quantity and layout of one or more magnets generating the field. In some embodiments, more than one magnet is utilized to align the magnetic field lines in a specific orientation with respect to gas flow paths. When a mixture of paramagnetic and non-paramagnetic gases flows through a tube, the gas mixture becomes inhomogeneous, with paramagnetic gas molecules being drawn to one side of the gas flow path. Once drawn to one side of gas flow path, the paramagnetic gas molecules can be separated from the main stream (e.g., through a membrane, or through a bifurcation in the flow path.

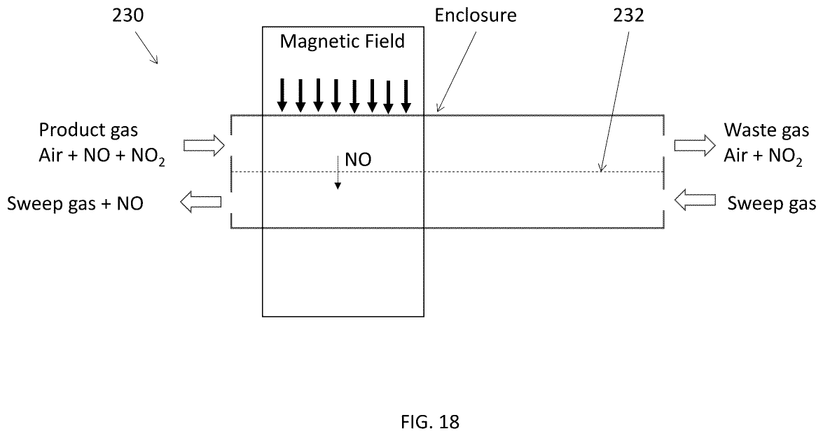

It can be possible to use a membrane in conjunction with a magnetic field to achieve selective movement of gas molecules within a gas flow to separate components of a gas flow. FIG. 18 depicts an embodiment of a membrane gas scrubber 230 whereby a membrane 232 preferentially permits passage of NO vs. NO₂. In some embodiments, preferential passage of NO through a membrane is accomplished by pore size. The scrubber shown in FIG. 18 can also include an applied magnetic field that propels NO molecules through the membrane due to the paramagnetic properties of NO. Since NO₂ is not paramagnetic, it is not directed towards the membrane.

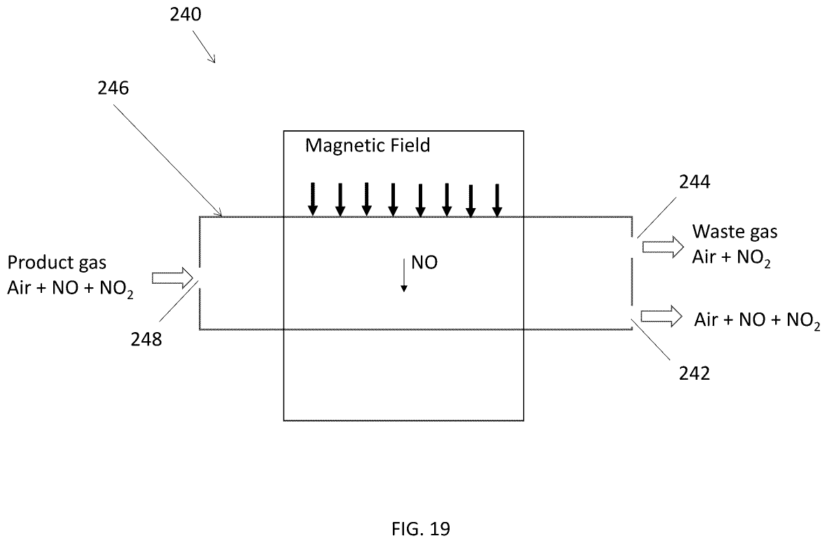

The placement, orientation, strength, and number of magnets used with a membrane can vary depending on the desired flow of gas and which components of the gas flow are required to pass through the membrane. FIG. 19 depicts an exemplary embodiment of a membrane gas scrubber 240 with a housing 246 where a magnetic field is applied across the direction of flow of a NO-containing gas stream. A product gas comprising air, NO, and/or NO₂ enters the housing through an inlet 248. The magnetic field directs NO towards a first outlet 242 so that the concentration of NO will be higher at the first outlet 242 than a second outlet 244. For maximum effect, the magnetic field is orthogonal to the gas direction of travel. This approach to gas separation allows for low flow restriction and no particle generation. Gas flow rate through this and similar gas separation devices must be laminar to ensure gas separation.

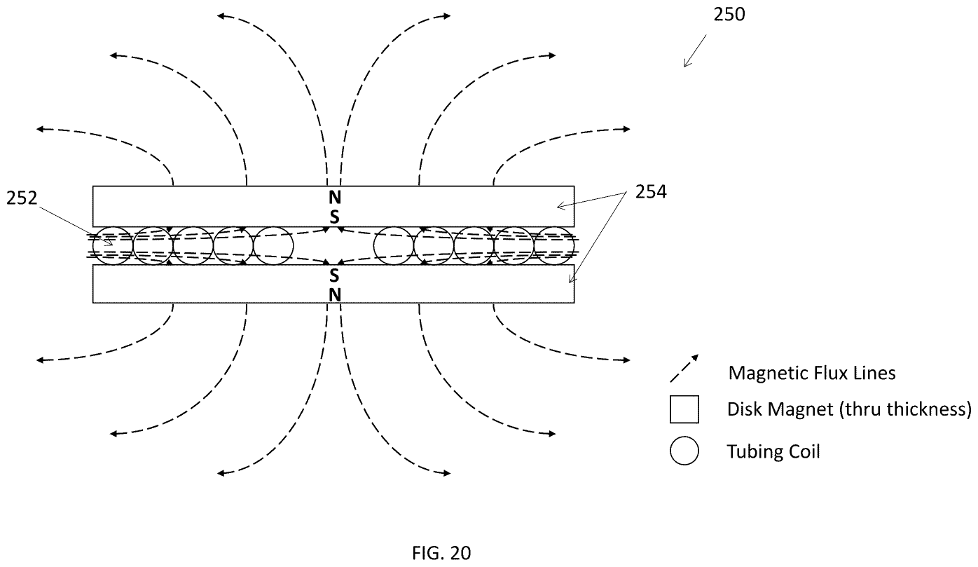

FIG. 20 depicts a side cross-section view of an exemplary embodiment of a magnetic field gas separation device 250 where a product gas stream is passed through a spiral-shaped gas flow path within a magnetic field. Product gas containing NO and NO₂ enters the assembly in the center (e.g., through a hole in one of the magnets) and flows through the spiral-shaped flow tube 252. The spiral tube is located between two parallel opposed magnets 254, such as magnetic discs. The opposed magnetic discs repel each other and require a frame to hold them in place (not shown). The magnetic field causes the paramagnetic portions of the gas to migrate towards the outer edges of the tubing. As the gas exits the magnetic field, or just before the exit, the gas passes by a divider that separates the outer and inner layers of gas flow. Depending on the direction of the magnetic field, either the outer or the inner layer of flow will have higher NO concentration. The other layer of flow will contain higher contents of nitrogen dioxide and other non-paramagnetic gases. Use of two magnets helps direct the field in a consistent direction with respect to the gas flow. A similar approach can be done with a magnet on one side of the spiral. In another embodiment, the gas flow path is in the form of a constant radius coil with one or more revolutions within the magnetic field.

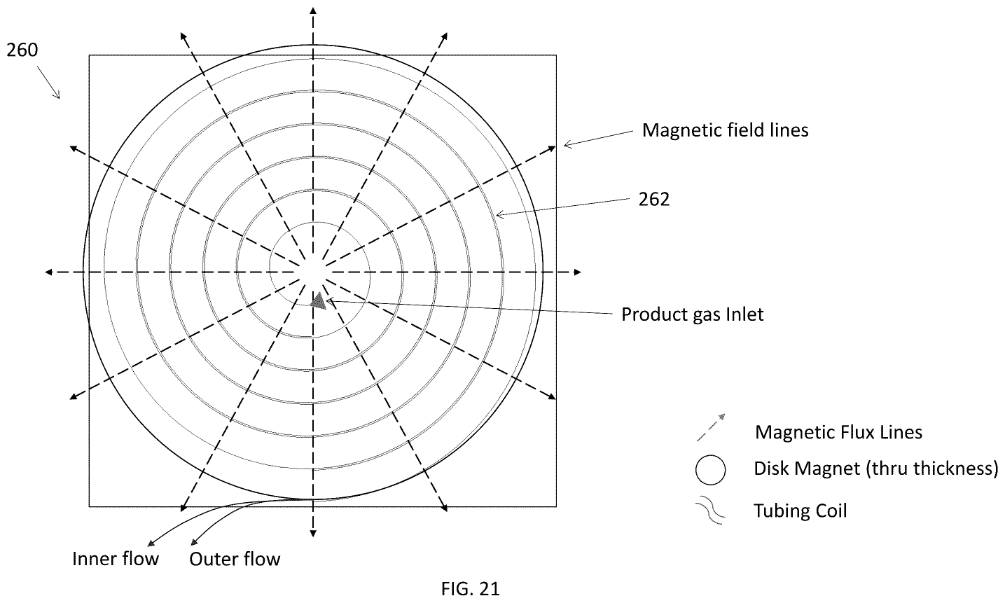

FIG. 21 depicts a top-down cross-sectional view of a magnetic field gas separation device 260 that includes a spiral-shaped coil 262 for the flow path of the product gas with magnetic field for separating a gas stream. The number of spiraled product gas flow coils or tubes can vary. In some embodiments, multiple spiral product gas tubes are utilized in parallel. This can enable a smaller diameter tube to be used for a given product gas flow rate, permitting the magnets to be located closer together for stronger magnetic field. Radiating lines depict the magnetic field lines. Paramagnetic gases within the product gas flow stream travel to the outer surface of the coil under the influence of the magnetic field. As the product gas exits the coil assembly, the gas is separated into an inner gas stream and an outer gas stream, the outer gas stream containing a higher concentration of paramagnetic gas (e.g., NO).

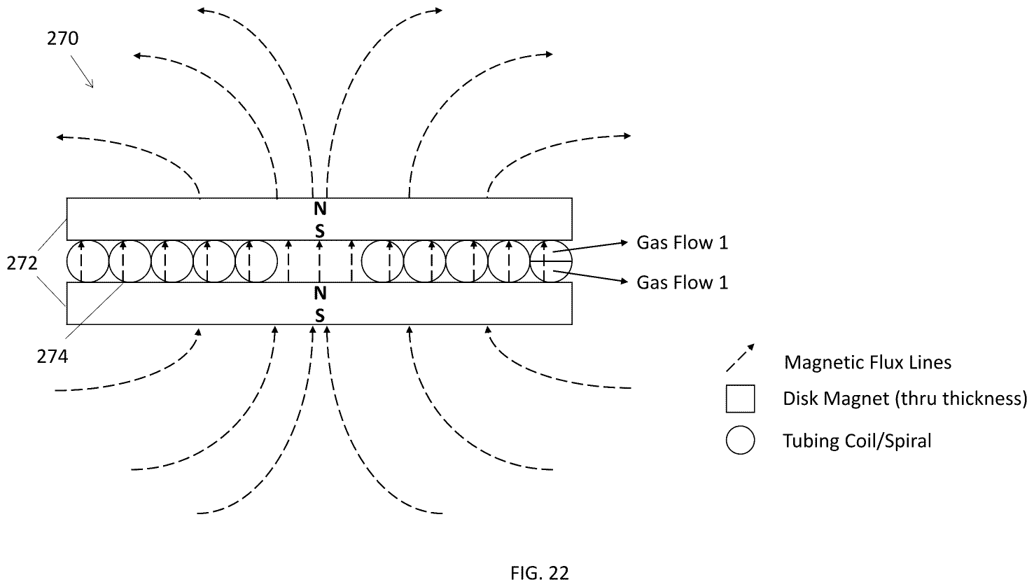

FIG. 22 depicts a magnetic field gas separation device 270 that utilizes magnets 272 that are aligned with their polarity in the same direction. This generates a magnetic field across the gap between the magnets, directing paramagnetic gases along the magnetic field to one side of a product gas tube 274. At the exit of the coiled or spiraled product gas path, the product gas flow is separated into upper and lower (with respect to the diagram) gas flows to separate the paramagnetic gas (e.g., NO) from other gases.

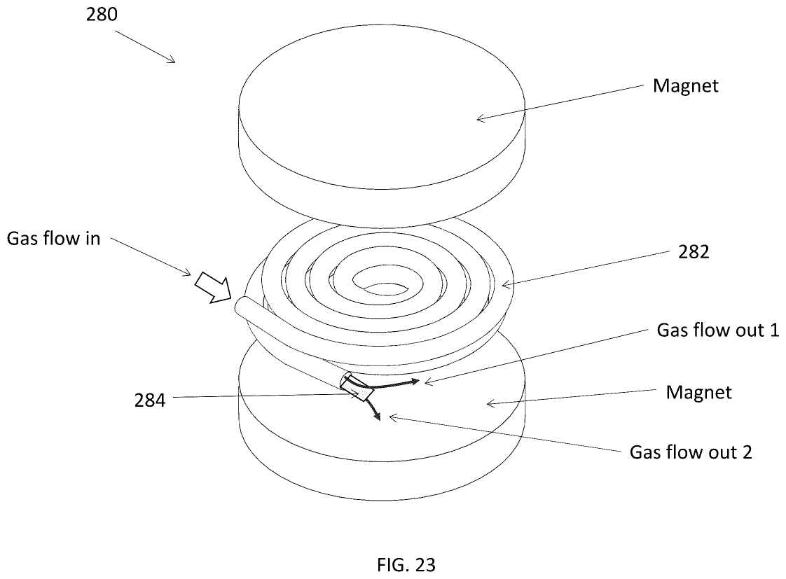

FIG. 23 depicts an exploded isometric view of an exemplary embodiment of a magnetic field gas separation device 280 where a gas flow tube 282 carrying the product gas enters the assembly tangentially, flowing into a converging section of spiraled tube. At the innermost coil, the tube continues to wind in the same spiral direction but offsets to a second plane where it continues to spiral in a diverging pattern. As the gas reaches the end of the flow path, it flows through a gas separator 284 that separates the strata of gas flow into one or more outbound streams (two streams shown). The layers of gas within the tube are sequenced according to the magnetic field with the most paramagnetic gases on one side of the flow conduit and non-paramagnetic gases on the opposite side of the flow conduit. This design allows for a longer flow path through the magnetic field to prolong gas separation time, and it can be assembled with solid magnets that do not have a hole, simplifying the magnet design.

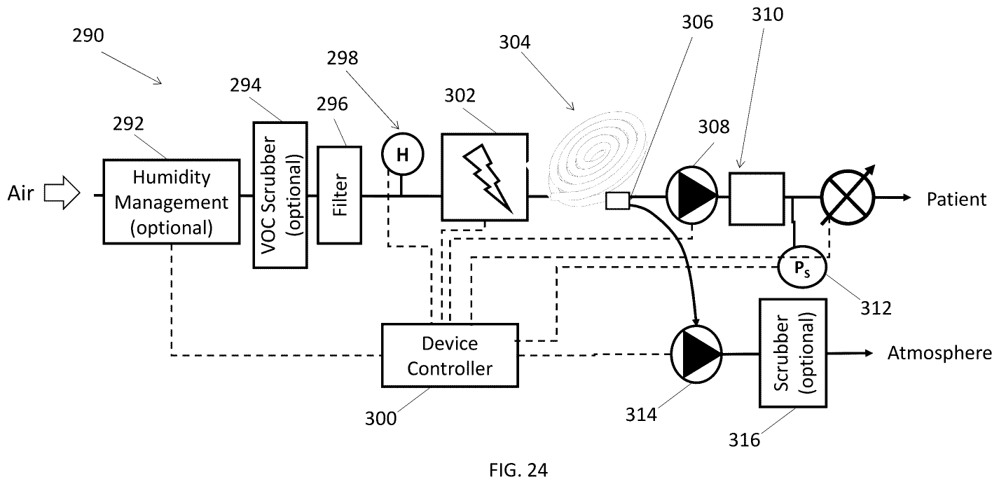

FIG. 24 depicts an exemplary embodiment of a NO generation system 290 with a magnetic gas separator. Atmospheric air enters the system passes through an optional humidity management subsystem 292, optional VOC scrubber 294, and a particle filter 296. Humidity of the incoming gas is optionally measured with a humidity sensor 298. The humidity measurement is read by a controller 300 (e.g., programmable logic controller, FPGA, etc.) that utilizes the humidity level as feedback to control the optional humidity management system. Furthermore, the controller utilizes the humidity measurement as input to an algorithm to determine plasma activity levels (e.g., energy, duty cycle, frequency, dithering) required to achieve a target NO concentration in the product gas. A portion of the diatomic nitrogen and diatomic oxygen in the air are converted to NO within a plasma chamber 302. The gas then flows through a magnetic gas separation device 304 that draws NO to one side of the spiral gas flow path. The magnetic gas separation device can utilize any of the embodiments described herein. As gas exits the magnetic gas separate device, it passes through a gas flow splitter 306 that divides the flow into a first gas flow containing NO and a second gas flow. The first gas flow is drawn through a pump 308 that pressurizes an optional reservoir 310 with pressure measured via an optional sensor 312. In some embodiments, the gas reservoir includes NO₂ scrubbing material (e.g., soda lime). NO-containing gas is controllably released from the reservoir by a flow controller, as directed by the device controller. The second gas flow exiting the gas splitter is also drawn by a pump 314. The relationship between pump speed between the first and second pumps 308, 314 determines the portion of product gas entering the first flow vs. the second flow. Gas exiting the second pump passes through an optional scrubber 316 (e.g., soda lime, ascorbic acid, MOF, activated carbon, potassium permanganate) and is released into the atmosphere. In some embodiments, the secondary gas flow is released to a house exhaust system or other environment in which scrubbing is not required.

In some embodiments, this ratio of flow between the first gas flow and the second gas flow is varied according to one or more of the quantity of NO within the product gas, patient demand for NO, pressure within the optional reservoir, and other parameters. For example, in some embodiments, the sum of flow rates for the two pumps is the same at all times. This ensures consistent reactant gas flow rate within the plasma chamber and controlled, laminar flow through the magnetic gas separator. In some embodiments, when the optional reservoir pressure reaches a threshold level, the first pump flow rate slows or stops and the second pump increases in flow rate, at the direction of the device controller, to maintain a constant reactant gas flow rate.

### NO₂ Condensation

In some embodiments, NO product gas is cooled to below the NO₂ boiling point to induce condensation (21.1° C.). The condensed NO₂ takes on the form of N₂O₄, a heavy molecule. When combined with centripetal separation techniques, N₂O₄ droplets as well as condensed water collect at the periphery of a gas separation vessel. These liquid constituents can be drained from the vessel to separate water and NO₂ from the remaining gas. In some embodiments, scrubbed product gas is withdrawn from the center of the centripetal chamber, where the lighter gases (e.g., N₂ and NO) collect.

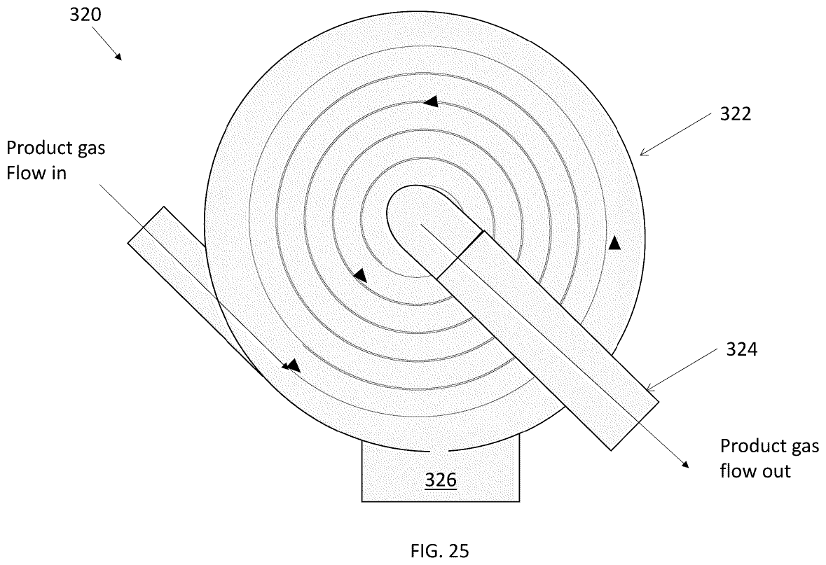

FIG. 25 depicts an embodiment of a centripetal gas separator 320. The gas separator consists of a housing 322 with a circular inner chamber. A flow of product gas is introduced to the chamber tangentially. The gas flow spirals through the chamber, exiting the chamber through an outlet 324 in the center of the chamber. In some embodiments, the housing is cooled to enhance gas and/or water vapor condensation. In some embodiments, internal features (e.g., curved walls, ridges, etc.) within the chamber increase heat transfer from the gas to the chamber, reducing the gas temperature further. As constituents in the gas stream condense (e.g., water, NO2), liquid particles are flung to the outer walls by centripetal acceleration, whereby they collect and flow down the walls of the separator by gravity to a collection trap 326 at the bottom of the device.

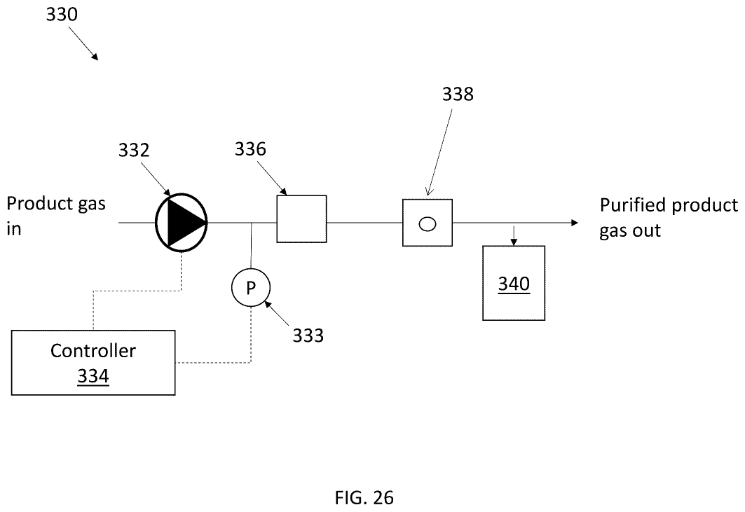

FIG. 26 depicts an exemplary embodiment of a NO generation system 330 that utilizes the Hampson-Linde process, a method for generating liquified gas, to separate water and/or NO₂ from a NO product gas stream. NO product gas enters the system and passes through a pump 332. The pump pressurizes the gas resulting in an increase in temperature. The pressure that the gas reaches is actively measured by a pressure sensor 333 and varied by a controller 334 that can vary pump activity. The gas is then cooled to a lower temperature by passing through a heat exchanger 336. The high pressure gas then travels through an orifice 338 (e.g., a critical orifice, flow controller, etc.). As the gas pressure drops on the downstream side of the orifice, so too does the gas temperature, causing condensation of some constituents in the gas stream. In some embodiments, the approach is utilized to remove NO₂ and/or water from a gas stream. Condensed gases are collected in a water trap 340 (e.g., centripetal trap, gravity trap, etc.)

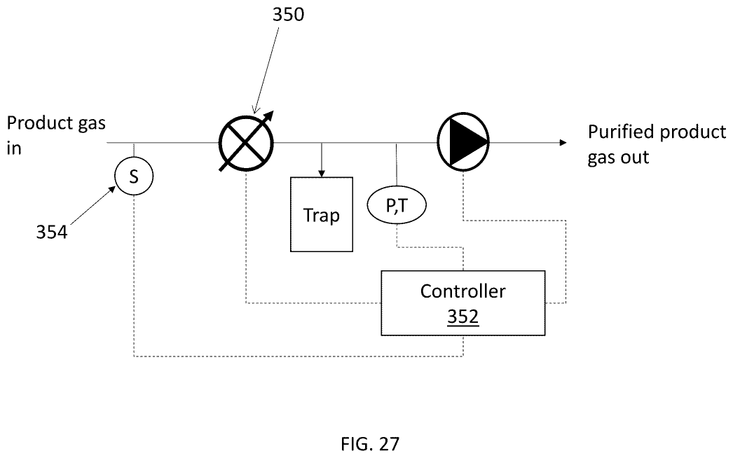

The rate of nitric oxide oxidation increases with pressure. FIG. 27 depicts an embodiment of a NO₂ and/or water separation process that utilizes vacuum pressure instead of high pressure. Product gas enters the system and is pulled through a flow restriction 350. As depicted, the flow restriction is variable, controlled by a controller 352, to vary the degree of gas separation. In some embodiments (not shown), the flow restriction is a fixed orifice. An optional sensor 354 (labeled “S”) in fluid contact with the incoming gas stream measures the quantity of a contaminant in the product gas stream (e.g., NO₂, humidity) to inform the controller as to the degree of gas separation required. For example, if the product gas already has acceptable amounts of water, the flow restriction can open and not restrict the gas flow.

The gas flow is pulled through the system with a pump. The pump effort is modulated by the controller, based on a pressure measurement upstream of the pump to maintain a target temperature and/or pressure within the product gas to ensure condensation of one or more constituents in the product gas. In some embodiments (not shown), the temperature of the product gas is measured downstream of the flow controller to inform the controller of the status of the process. Condensed gases can be collected in a water trap or other storage device.

### Environmental Considerations

The quantity of NO₂ to dispose of varies with the NO generation device and the patient treatment, but an exemplary estimate of high NO₂ production can be obtained as follows:
Patient treatment: 80 ppm NO in 30 lpm of inspiratory gas
NO production at patient: 2400 ppm·lpm
NO: NO₂ ratio: 10:1
NO₂ production: =240 ppm·lpm

In some embodiments, the NO generation system releases the NO₂-laden sweep gas directly into the atmosphere. This is not an issue in the outdoors, where the NO₂ output of a medical NO device is orders of magnitude less than the limitations set by EPA for automobiles (˜1000 ppm NOx@1000 lpm, or 1,000,000 ppm·lpm for a 2 liter vehicle at idle).

Release directly to the environment is unlikely to present a safety concern in the hospital environment as well, where tank-based NO systems dose ventilatory flows and excess gas not inhaled by the patient is discharged into the environment. This is usually not an issue because a hospital environment typically exchanges the air with each room multiple times per hour. As a point of reference, a tank-based NO delivery system delivering 80 ppm NO to a 30 lpm flow will result in 2000 ppm·lpm of wasted NO entering the room (2400 ppm·lpm total production minus 5 lpm minute volume*80 ppm NO inhaled by the patient). An electric NO system with a 20:1 NO/NO₂ ratio would produce 4 ppm of NO₂ at 30 lpm, or 120 ppm·lpm of NO₂. Assuming the scrubber to be 100% efficient, that 120 ppm·lpm of NO₂ would pass through the membrane and enter the atmosphere in addition to 2000 ppm·lpm of NO₂ from wasted NO (same as the tank scenario above). This would be a 6% increase (120/2000=6%) in NO₂ over the tank-based equivalent treatment, assuming that all NO introduced to the environment eventually becomes NO₂.

In some applications, it is not acceptable to exhaust NO₂-laden air into the environment. This could be the case for small rooms or rooms with less air exchange. In this case, the sweep gas can be directed to the vacuum system of the facility. In some embodiments, the sweep gas passes through a separate NO/NO₂ scrubber prior to release into the atmosphere. In some embodiments, the NO/NO₂ scrubber is replaceable. Example chemistries include one or more of TEMPO, activated carbon, soda lime, potassium permanganate, potassium hydroxide, calcium hydroxide, sodium hydroxide, metal organic frameworks, and ascorbic acid. In some embodiments, the sweep gas is chilled to make the NO₂ condense (boiling point is 21.5 deg C). In some embodiments, the product gas is cooled to a temperature, for example, below 21.5 deg C but above −152 deg C (the boiling point of NO) so that NO₂ condenses out of solution. Condensed NO₂ is then collected in liquid form for later disposal.

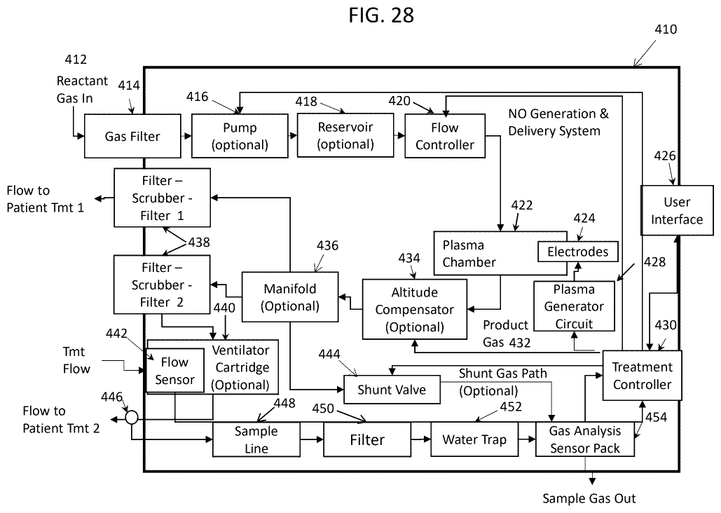

The gas separators described herein can be used with systems that are configured to generate a product gas containing NO. FIG. 28 illustrates an exemplary embodiment of a NO generation system 410 that includes components for reactant gas intake 412 and delivery to a plasma chamber 422. The plasma chamber 422 includes one or more electrodes 424 therein that are configured to produce, with the use of a high voltage circuit (plasma generator) 428, a product gas 432 containing a desired amount of NO from the reactant gas. The system includes a controller 430 in electrical communication with the plasma generator 428 and the electrode(s) 424 that is configured to control the concentration of NO in the product gas 432 using one or more control parameters relating to conditions within the system and/or conditions relating to a separate device for delivering the product gas to a patient (e.g. flow rates, pressures, gas concentrations (e.g. O₂, He)) and/or conditions relating to the patient receiving the product gas (e.g. SpO₂, tidal volume, clinical indication). In some embodiments, the plasma generator circuit is a high voltage circuit that generates a potential difference across an electrode gap.

In some embodiments, the NO system pneumatic path includes a pump pushing air through a manifold 436. In some embodiments, the manifold is configured with one or more valves: three-way valves, binary valves, check valves, and/or proportional orifices. The treatment controller 430 controls the flow of the pump, the power in the plasma and the direction of the gas flow post-electrical discharge. By configuring valves, the treatment controller can direct gas to the manual respiration pathway, the ventilator pathway or the gas sensor chamber for direct measurement of NO, NO₂ and O₂ levels in the product gas. In some embodiments, respiratory gas (i.e., the treatment flow) can be directed through a ventilator cartridge that measures the flow of the respiratory gas and can merge the respiratory gas with NO product gas. In some embodiments, product gas is directed to a remote injection module (not shown), where inspiratory gas mass flow rate is measured, and product gas is injected into the inspiratory gas flow.

The output from the NO generation system in the form of the product gas 432 enriched with the NO produced in the plasma chamber 422 can either be directed to a respiratory or other (e.g., external applicator) device for delivery to a patient, or can be directed to a plurality of components provided for self-test or calibration of the NO generation system. In some embodiments, the system collects gases to sample in two ways: 1) gases are collected from a patient inspiratory circuit near the patient and pass through a sample line 448, a filter 450, and a water trap 452, or 2) gases are shunted directly from the pneumatic circuit as they exit the plasma chamber 422. In some embodiments, product gases are shunted with a shunt valve 444 to the gas sensors after being scrubbed but before dilution into a patient airstream. In some embodiments (not shown), shunted product gas is diluted to reduce the concentration before delivery to gas sensors. In some embodiments, product gases are collected from an inspiratory air stream near the device and/or within the device post-dilution. Within the gas analysis portion of the device, the product gas passes through one or more sensors to measure one or more of temperature, humidity, concentrations, pressure, and flow rate of various gasses therein.

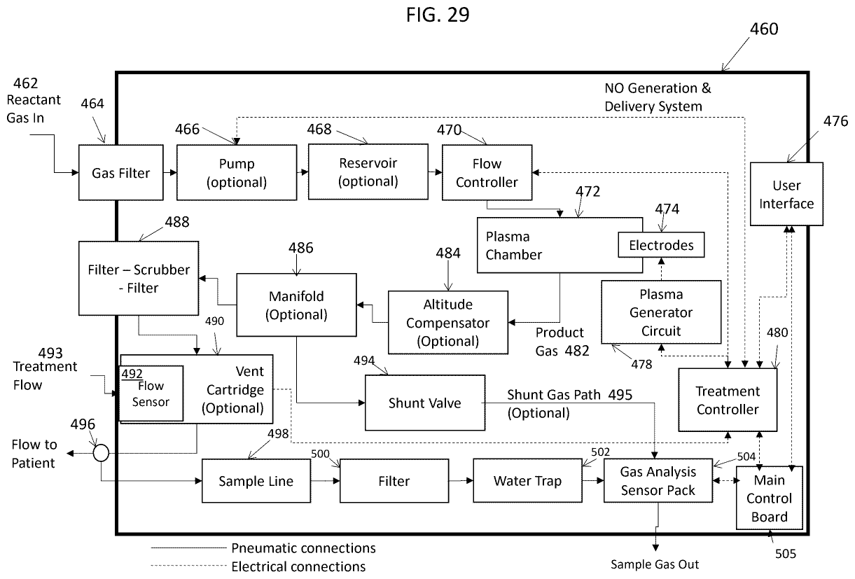

FIG. 29 depicts an embodiment of a NO generation and delivery system 460. Reactant gas 462 enters the system through a gas filter 464. A pump 466 is used to propel gas through the system. Whether or not a system includes a pump can depend on the pressure of the reactant gas supply. If reactant gas is pressurized, a pump may not be required. If reactant gas is at atmospheric pressure, a pump or other means to move reactant gas through the system is required. A reservoir 68 after the pump attenuates rapid changes in pressure and/or flow from a pump. Coupled with a flow controller 470, the reservoir, when pressurized, can enable a system to provide flow rates to the plasma chamber 472 that are greater than the pump 466 flow rate. Electrodes 474 within the plasma chamber 472 are energized by a plasma generation circuit 478 that produces high voltage inputs based on desired treatment conditions received from a treatment controller 480. A user interface 476 receives desired treatment conditions (dose, treatment mode, etc.) from the user and communicates them to the main control board 505. The main control board 505 relays to the treatment controller 480 a target dose and monitors measured NO concentrations from the gas analysis sensor pack 504. The main control board 505 monitors the system for error conditions and can generate alarms, as required. Reactant gas 462 is converted into product gas 482 when it passes through the plasma chamber 472 and is partially converted into nitric oxide and nitrogen dioxide. An altitude compensator 484, typically consisting of one or more valves (i.e., proportional valves, binary valves, 3-way valves, etc.), is optionally used to provide a back-pressure within the plasma chamber 472 for additional controls in nitric oxide production. Product gases pass through a manifold 486, as needed, to reach a filter-scavenger-filter 488 assembly that removes nitrogen dioxide and/or particulates from the product gas. From the filter-scavenger-filter 488, product gas is introduced to a patient treatment flow directly, or indirectly through a vent cartridge 490. In some embodiments, the vent cartridge 490 includes a flow sensor 492 that measures the treatment flow 493. The treatment flow measurements from the flow sensor 492 serve as an input into the reactant gas flow controller 470 via the treatment controller 480. After product gas 482 is introduced to the treatment flow, it passes through inspiratory tubing. Near the patient, a fitting 496 is used to pull a fraction of inspired gas from the inspiratory flow, through a sample line 498, filter 500, water trap 502 and Nafion (or equivalent) tubing to prepare the gas sample and convey it to gas sensors 504. The Nafion tubing adds ambient humidity to the gas sample when dry calibration gas is used and removes water from the same when humid gas samples are collected to protect the gas sensors from gas humidity levels that are out of range. Sample gas exits the gas analysis sensor pack 504 to ambient air. In some embodiments, the system 460 can optionally direct gas through a shunt valve 494 and shunt gas path 495 directly to the gas sensor pack and out of the system. In some embodiments involving the shunt valve 494, the manifold 486 includes a valve (not shown) to block flow to the filter-scavenger-filter when the shunt valve 494 is open. In some embodiments (not shown), the shunted product gas is diluted with non-NO containing gas prior to delivery to the gas sensors.

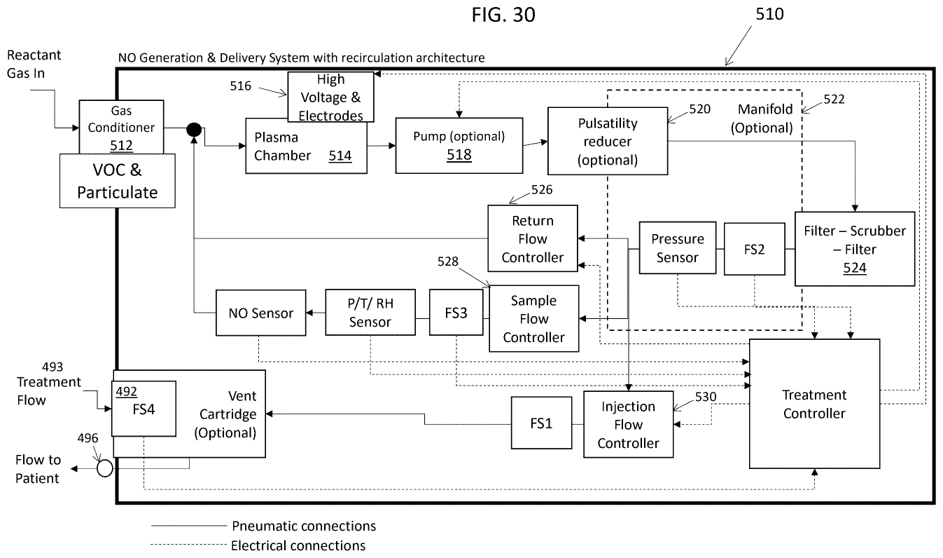

FIG. 30 illustrates an embodiment of a NO system with a recirculation architecture, a NO system design in which a portion of product gas is injected into an inspiratory stream and a portion is not injected. FIG. 30 depicts an embodiment of a NO generation and delivery system 510 that utilizes a recirculation architecture with reactant gas entering the system and passing through a gas conditioner 512 containing one or more of a particulate filter, VOC scrubber (e.g. activated charcoal), desiccant (e.g. molecular sieve, silica gel), and NO₂ scrubber (e.g. soda lime). In some embodiments (not shown), reactant gas temperature, pressure and humidity are measured by sensors located in the reactant gas pathway. Gas flows to a plasma chamber 514 where high voltage is applied to electrodes 516 to generate nitric oxide product gas. Product gas passes through a pump 518 and on through an optional pulsatility reducer 520 to decrease fluctuations in the pressure and/or flow rate of the product gas. A dashed line encloses components that can be part of or attach to a manifold 522 to simplify pneumatic routing. After passing through the pulsatility reducer, product gas passes through a filter/scrubber/filter 524. The filter/scrubber/filter removes particulate and NO₂ from the product gas. It should be noted that some scrubbers (e.g., one using sheet material) do not include one of the filters in some embodiments due to the lack of scrubber particulate generated. The scrubber can be any of the embodiments described herein, including gas separators with or without magnetic components. In some embodiments, the filter/scrubber/filter is user replaceable. From the filter/scrubber/filter, pressure and flow of the product gas is measured prior. Then, the product gas is divided into one to three separate flow paths. In one path, product gas flows through a return flow controller 526, back to before the plasma chamber. In another path, product gas flows through a sample flow controller 528, flow sensor, pressure sensor, temperature sensor, humidity sensor, and NO sensor. In another path, product gas flows through an injection flow controller 530 and flow sensor prior to being injected into a treatment flow of gas. Gas flowing through the return path merges with incoming reactant gas prior to entering the plasma chamber. In some embodiments, the plasma chamber is at or near atmospheric pressure. In some embodiments, the pressure within the plasma chamber is below atmospheric pressure, due to the flow restriction of the inlet filter/scrubber. Lower pressure within the chamber can reduce break-down voltage requirements and enable low levels of NO production. The return flow controller is modulated to maintain a constant pressure within the tubing upstream of the flow controllers while the sample flow controller maintains a target flow rate for the product gas NO sensor and the injection flow controller releases product gas at a target flow rate. In some embodiments, the target injection flow rate is proportional to the treatment flow. A constant pressure upstream of the injection flow controller improves flow control and dose accuracy. A treatment controller communicates with various components in the system, including various sensors, the injection flow controller, and the high voltage circuit and electrodes.

All patents, patent applications, and published references cited herein are hereby incorporated by reference in their entirety. It will be appreciated that several of the above-disclosed and other features and functions, or alternatives thereof, may be desirably combined into many other different systems or application. Various alternatives, modifications, variations, or improvements therein may be subsequently made by those skilled in the art.

What is claimed is:

1. A nitric oxide (NO) delivery system, comprising:
a plasma generating device configured to produce a plasma to ionize a flow of a reactant gas into a product gas, the product gas comprising NO, NO₂, oxygen, and nitrogen gases;
a controller configured to regulate an amount of NO in the product gas using one or more parameters as input to the controller, the one or more parameters including information related to at least one of the reactant gas, the product gas, a flow of gas into which the product gas is configured to be delivered, and a sweep fluid for removing scrubbed gases from the product gas; and
a gas separation device comprising:
a housing including one or more product gas inlets and one or more sweep fluid inlets, the one or more product gas inlets being configured to receive a flow of the product gas, the one or more sweep fluid inlets being configured to receive a flow of the sweep fluid such that the flow of product gas and the flow of the sweep fluid are opposed flows; and
at least one membrane positioned inside the housing, the at least one membrane configured to permit flow of a subset of gases of the product gas therethrough such that the product gas exiting the housing includes NO, wherein the flow of the sweep fluid through the housing is configured to move a subset of gases separated from the product gas away from the at least one membrane.

2. The system of claim 1, wherein the sweep fluid is configured to pass through a filter before entering at least one of the one or more sweep fluid inlets of the housing of the gas separation device.

3. The system of claim 1, wherein the sweep fluid is configured to exit the housing through an outlet, the sweep fluid configured to pass through a filter after exiting the housing of the gas separation device.

4. The system of claim 1, further comprising one or more pumps upstream of the housing configured to apply a pressure gradient across the at least one membrane to promote gas transport across the at least one membrane.

5. The system of claim 1, wherein the at least one membrane comprises a plurality of tubular membranes to provide a plurality of product gas pathways through the gas separation device.

6. The system of claim 5, wherein the plurality of tubular membranes are positioned in parallel to provide even flow restriction therethrough.

7. The system of claim 1, wherein a flow of product gas exiting the housing is configured to flow to one of the one or more product gas inlets to provide a recirculation flow of product gas through the gas separation device.

8. The system of claim 1, wherein a flow of the sweep fluid exiting the housing is configured to flow to one of the one or more sweep fluid inlets to provide a recirculation flow of the sweep fluid through the gas separation device.

9. The system of claim 1, wherein the sweep fluid is reactant gas.

10. The system of claim 1, wherein the plasma generating device includes at least one pair of electrodes.

11. The system of claim 10, wherein the controller is configured to regulate the amount of NO in the product gas by using the one or more parameters to control sparking of the at least one pairs of electrodes.

12. The system of claim 1, wherein the controller is configured to regulate the amount of NO in the product gas by using the one or more parameters to control energy transferred to the plasma.

13. The system of claim 1, further comprising a sensor configured to measure a flow of the sweep fluid, the sensor configured to communicate the measured flow of the sweep fluid to the controller such that the controller suspends plasma production when the measured flow of the sweep fluid is below a threshold.

14. The system of claim 1, wherein the gas separation device is in the form of a replaceable cartridge.
